(19) United States
(12) Patent Application Publication
(10) Pub. No.: US 2024/0060966 A1
(43) Pub. Date: Feb. 22, 2024

(54) OPTICAL MODULE

(71) Applicant: ams International AG, Jona (CH)

(72) Inventors: Boon Chong CHEAH, Geldrop (NL); Remco VERDOOLD, Geldrop (NL); Filip FREDERIX, Molenstede (BE); Erik Jan LOUS, Veldhoven (NL)

(21) Appl. No.: 18/267,301

(22) PCT Filed: Dec. 13, 2021
(86) PCT No.: PCT/EP2021/085546

§ 371 Date: Jun. 14, 2023

(30) Foreign Application Priority Data
Dec. 15, 2020 (GB) 2019772.9

(51) Int. Cl. G01N 33/52; G01N 21/78; B01L 3/00; G01N 33/543

(52) U.S. Cl. CPC G01N 33/521; G01N 21/78; B01L 3/5023; G01N 33/54388; G01N 2021/7759

(57) ABSTRACT

An optical module for reading a test region of an assay includes a near-infrared light source for illuminating the test region of the assay with light in a near-infrared spectrum. The optical module also includes an optical detector. The optical detector includes an optical input for receiving light emitted from the test region of the assay and an electrical output. The optical module further includes an electrical signal processor electrically coupled to the electrical output. The optical module additionally includes one or more optical filter arranged in front of the optical input of the optical detector.

### TECHNICAL FIELD

The present invention relates to assay readers, in particular but not limited to optical modules for assay readers.

### BACKGROUND

Diagnostic tests are commonly used for identifying diseases. A diagnostic test may be carried out in a central laboratory, whereby a sample, for example blood, is taken from a patient and sent to the central laboratory where the sample is analysed. A different setting for processing samples is at the point where care for the patient is delivered, which is referred to as point-of-care (POC) tests. POC tests allow for a faster diagnosis. Within the POC tests, different technology platforms can be used. A first class of POC tests are high end, microfluidic-based POC tests. These POC tests are mainly used in a professional environment such as hospitals or emergency rooms. A different technology platform is provided by lateral flow test technology. Some lateral flow tests are used in the consumer area, such as for pregnancy tests, and are easy to produce and very cost-effective.

Lateral flow tests are very well known as such, but are briefly described by way of background. A lateral flow assay includes a series of capillary beds, such as pieces of porous paper, nitrocellulose membranes, microstructured polymer, or sintered polymer for transporting fluid across a series of pads by capillary forces. A sample pad acts as a sponge and is arranged to receive a sample fluid, and further holds an excess of the sample fluid. After the sample pad is saturated with sample fluid, the sample fluid migrates to a conjugate pad in which the manufacturer has stored the so-called conjugate. The conjugate is a dried format of bio-active particles in a salt-sugar matrix intended to create a chemical reaction between the target molecule (e.g., an antigen) and its chemical partner (e.g. antibody or receptor). While the sample fluid dissolves the salt-sugar matrix, it also mobilizes the bio-active particles and in one combined transport action the sample and conjugate mix with each other while flowing through the capillary beds. The analyte binds to the particles while migrating further through the third capillary bed. This material has one or more areas, which are called stripes, where a third type of molecule has been immobilized by the manufacturer, in most cases an antibody or receptor addressed against another part of the antigen. By the time the sample-conjugate mix reaches these strips, analyte has been bound on the particle and the third type of molecule binds the complex. When more fluid has passed the stripes, particles accumulate on the stripes and the stripes become visible, appear or are generated in a particular colour or with a fluorescent wavelength capability. In this way the stripe is optically detectable by colour or by fluorescent emission detection, respectively.

Typically, there are at least two stripes: a control stripe/line that captures the conjugate and thereby shows that reaction conditions and technology work, and a second stripe, the test stripe/line, that contains a specific capture molecule and only captures those particles onto which an analyte or antigen molecule has been immobilized. This makes the diagnostic result of the test visible for the patient. Some test results rely on the presence of fluorescent particles, which may not be visible to the user but can instead be detected by optical detectors when the stripes are illuminated. After passing the different reaction zones, the fluid enters the final porous material, which is a wick that acts as a waste container.

The lateral flow test strip can contain multiple test lines, where each test line contains a different type of specific capture molecule, which binds to a different analyte or antigen. This multi-analyte detection, using spatially separated test lines, can be done using the same colour or fluorescent emission wavelength for the optical detection. However, each test line can also be made visible by different colours or fluorescent emission wavelength. For example, each type of specific receptor bound to its respective analyte-conjugate complex may have a different colour or emission wavelength. Ultimately, these test lines can be one line, not spatially separated, on the lateral flow test strip, but can be spectrally separated by the different colours or emission wavelengths.

In summary, lateral flow tests as such are well known and have four key elements: the antibody, the antigen, the conjugate and the complex. Despite these key elements being well established, the terminology used by the skilled person is not always consistent and different terms may refer to the same element. The antibody is also referred to as receptor, chemical partner, or capture molecule. The antigen is also referred to as analyte, target molecule, antigen molecule, target analyte or biomarkers. The sample typically contains the analyte, although that is not always the case. The conjugate is also referred to as (analyte) tags, tagging particles, chemical partner, (sample) conjugate mix, bioactive particles or conjugate receptors. Examples of conjugates are fluorescent particles, red particles or dyes, and further examples are provided in the specific description. The complex is the combination of the antigen and conjugate. The complex is also referred to as tagged analyte, or particles onto which the analyte molecule has been immobilised.

Currently, there are lateral flow tests available that you can “read” with your naked eye. The most commonly known test is the pregnancy test that one can buy in a supermarket or pharmacy. These tests indicate if a certain analyte (e.g. HCG hormone in the case of a pregnancy test) is above a certain value or below a certain value. No quantification is possible and the sensitivity is limited to the colour difference one can observe with its naked eye. In addition, since it has to be visible with the naked, only conjugates (e.g. dyes) in the visible region can be used.

To increase the sensitivity and the level of quantification, electronic optical detectors exist where a lateral flow test strip is positioned with a light source and an optical detector. The electronic optical detector may be incorporated into a lateral flow assay device comprising the lateral flow test strip. Alternatively, the electronic optical detector may be incorporated into an assay reader device comprising an aperture for receiving a lateral flow assay device, the lateral flow assay device comprising the lateral flow test strip.

### SUMMARY

The inventors have identified that with known electronic optical detectors 1) the sensitivity is often hampered by the auto-fluorescent background signal and 2) multi-analyte detection is difficult, since additional light sources and detectors are needed in order to measure different kinds of analytes e.g. different kind of lines. In addition, many of the lateral flow tests use dyes in the visible wavelength band for detection since it is visible to the naked eye. However, many biological liquids and even the membrane have their autofluorescence in the visible region when excited by visible or ultraviolet (UV) light, which attributes to high background signal.

Fluorescence is an event whereby a substance absorbs energy from an electromagnetic radiation and emits light at a lower/higher energy than the absorbed energy. A lot of organic substances present on the fluids and/or the LFT-paper has fluorescence when it is excited with light, which is a natural occurrence, this is termed autofluorescence.

In a detection assay, a conjugate (e.g. an artificial fluorophore) is used to detect the concentration of a particular compound. However, autofluorescence of other natural compounds (such as human skin, blood, urine, plasma, nitrocellulose membrane etc.) also give out fluorescence at wavelengths that are the same as the desired detection. For instance, porphyrins in plasma autofluoresce at 590 nm and 630 nm, and nitrocellulose membranes autofluoresce at 500 nm and 600 nm. This in turns reduce the sensitivity, as it is considered as background noise with respect to the marker signal.

Embodiments of the present disclosure generally relate to an optical module which reduces the background autofluorescence when analysing a lateral flow test strip. This results in an increase of sensitivity by two to three orders of magnitude. This high sensitivity allows detection of biomarkers (and thus the corresponding diseases) previously not possible with eye-read lateral flow tests.

According to one aspect there is provided an optical module for reading a test region of an assay, the optical module comprising: a near-infrared light source for illuminating the test region of the assay with light in a near-infrared spectrum; an optical detector, comprising an optical input for receiving light emitted from the test region of the assay and an electrical output; an electrical signal processor, electrically coupled to the electrical output; and one or more optical filter arranged in front of the optical input of the optical detector.

This allows for very sensitive and quantitative measurements to be performed by the electrical signal processor.

The optical module may comprise a plurality of optical filters. This advantageously enables multiple analytes to be detected and measured without the need for additional lines or more detectors.

The plurality of optical filters may correspond to a plurality of spatially separated regions of the optical detector.

The optical detector may comprise an array of detectors, and wherein each detector of the array of detectors may correspond to each of said optical filters.

The one or more optical filter may be arranged in front of the optical input of the optical detector such that there is no spectral filter in front of a portion of the optical input. This enables the electrical signal processor to check the background light, to check the light intensity of the near-infrared light source, to check a reference area on the strip/membrane of the assay, or to check if there are an ambient light leaking into the detection system.

In some embodiments, the optical detector comprises the one or more optical filter.

The optical module may comprise a second near-infrared light source for illuminating a control region of the assay.

In some implementations, the optical filters are configured to enable the electrical signal processor to measure the absorption of the excitation wavelength due to the fluorescence properties of the conjugate used in the assay. In these implementations, the light emitted by the near-infrared light source has an excitation spectrum centred on an excitation wavelength and the one or more optical filter is transparent to at least a portion of said excitation spectrum, wherein said portion of the excitation spectrum is within an absorption spectrum of a conjugate used in said assay. The one or more optical filter may be configured to block light having wavelengths within an emission spectrum of said conjugate.

In other implementations, the optical filters are configured to enable the electrical signal processor to measure the emission of the fluorescence of the conjugate used in the assay. In these implementations, the light emitted by the near-infrared light source has an excitation spectrum centred on an excitation wavelength and the one or more optical filter is transparent to wavelengths within an emission spectrum of a conjugate used in said assay.

The wavelengths within the emission spectrum of the conjugate used in said assay may be at a higher wavelength than the excitation spectrum. Alternatively, the wavelengths within the emission spectrum of the conjugate used in said assay may be at a lower wavelength than the excitation spectrum. The fluorescence emission is too weak to trigger much autofluorescence, or it is negligible. In these implementations the optical module acts as an anti-stoke fluorescent reader. The wavelengths within the emission spectrum of the conjugate used in said assay may be in the visible spectrum or in the near-infrared spectrum.

In these other implementations the one or more optical filter may block light having wavelengths within the excitation spectrum of the infrared light source.

The optical module may further comprise a substrate for mounting the near-infrared light source and the optical detector. The electrical signal processor may be mounted on the substrate.

The substrate may comprise a printed circuit board of an assay reader device, or the substrate may be configured to be positioned on a printed circuit board of an assay reader device.

According to another aspect of the present disclosure there is provided an assay reader device comprising the optical module described herein.

The assay reader device may comprise a lateral flow test strip. That is, both the optical module and the lateral flow test strip may be packed into a single assay reader device. In these embodiments, the distance between the site of the colour change and the optical detector is very small which avoids a decreased signal or the need to use a more expensive or sensitive detector.

Alternatively the assay reader device comprises an aperture for receiving a lateral flow assay device comprising a lateral flow test strip. In these embodiments the lateral flow assay device comprising the lateral flow test strip is inserted into the aperture for the optical module to analyse the test lines on the lateral flow test strip.

According to another aspect of the present disclosure there is provided a method for reading a test region of an assay, the method comprising: illuminating the test region with a near-infrared light source that is operable to emit light in a near-infrared spectrum; providing the test region of the assay in the field of view of an optical detector; filtering light emitted from the test region using one or more optical filter to provide filtered light; and detecting the filtered light with the optical detector.

These and other aspects will be apparent from the embodiments described in the following. The scope of the present disclosure is not intended to be limited by this summary nor to implementations that necessarily solve any or all of the disadvantages noted.

### BRIEF DESCRIPTION OF THE PREFERRED EMBODIMENTS

Some embodiments of the disclosure will now be described by way of example only and with reference to the accompanying drawings, in which:

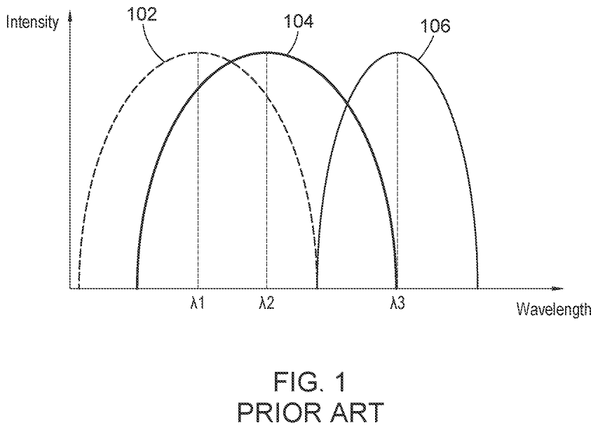

FIG. 1 illustrates a prior art technique of using visible light source is used to illuminate an assay;

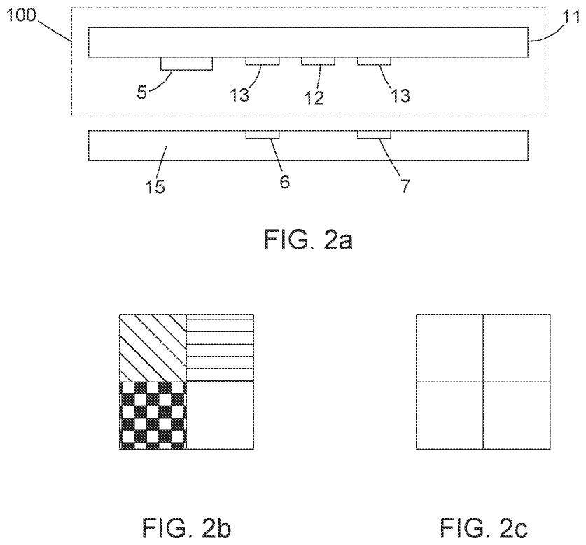

FIG. 2a is a schematic illustration of an optical module for reading an assay;

FIG. 2b illustrates optical filters which covers an optical detector of the optical module

FIG. 2c illustrates the optical detector behind the optical filters;

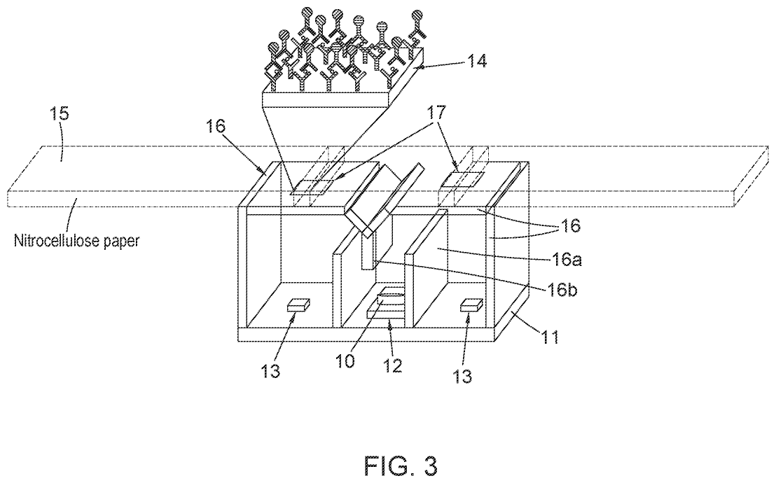

FIG. 3 is a perspective view of a schematic illustration of the optical module for reading an assay;

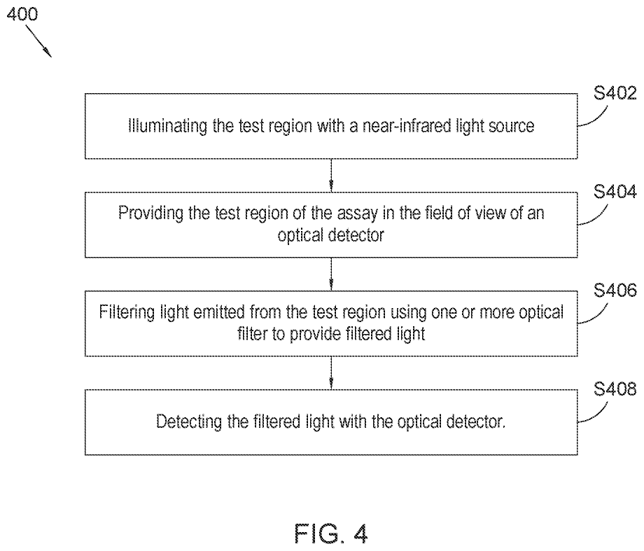

FIG. 4 is a flow diagram of a method;

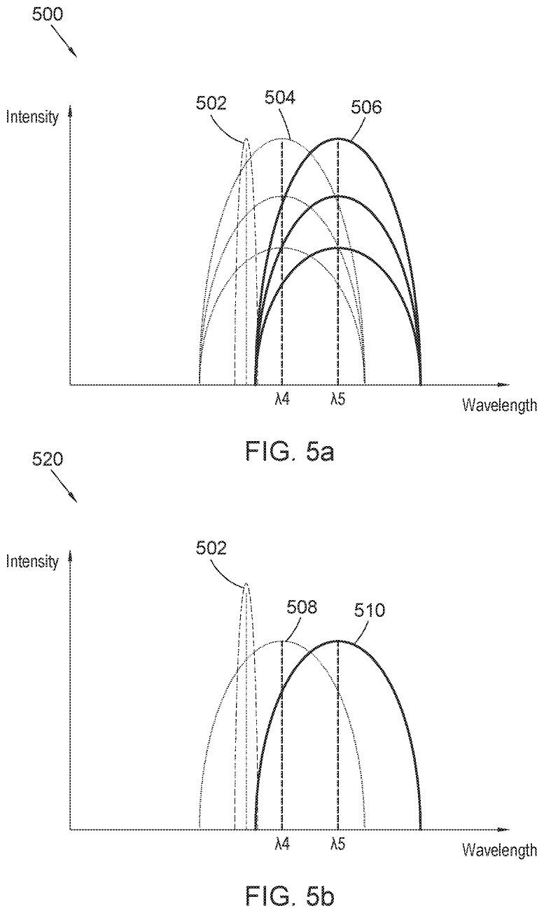

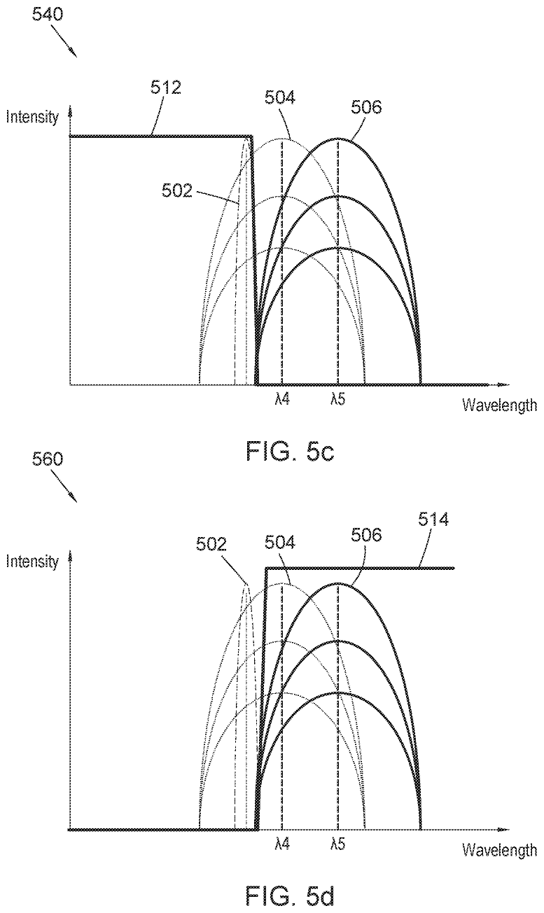

FIG. 5a shows an example plot associated with a test line of a lateral flow test strip;

FIG. 5b shows an example plot associated with a test line of a lateral flow test strip;

FIG. 5c illustrates an example filter response used in a first embodiment of the present disclosure;

FIG. 5d illustrates an example filter response used in a second embodiment of the present disclosure;

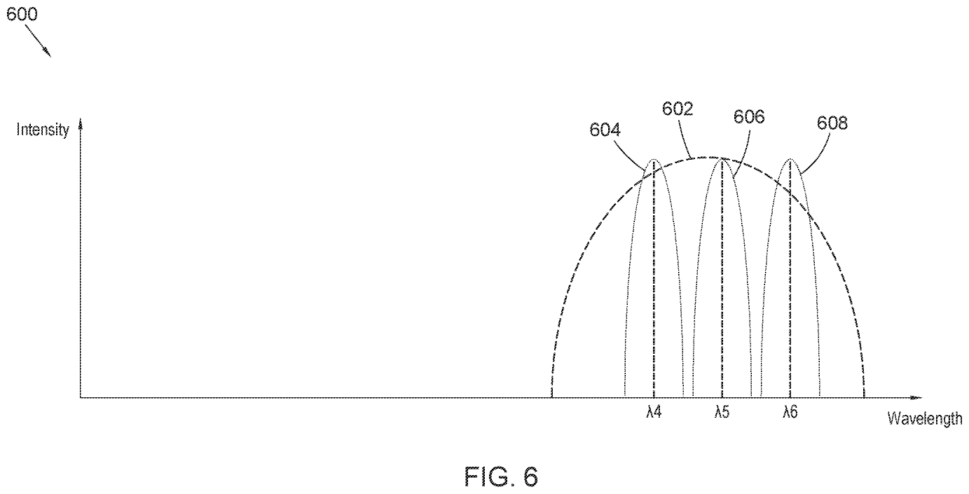

FIG. 6 illustrates multiple analyte detection according to the first embodiment of the present disclosure;

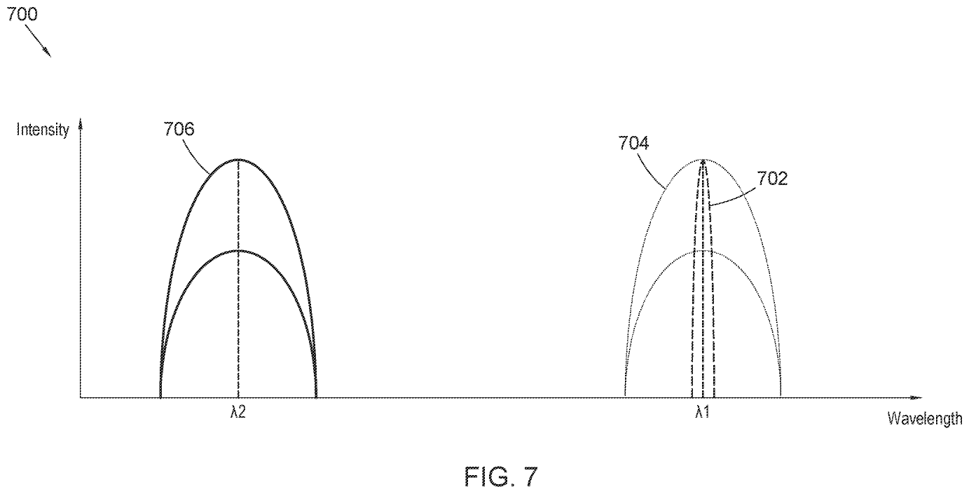

FIG. 7 shows an example plot associated with a test line of a lateral flow test strip in a third embodiment of the present disclosure;

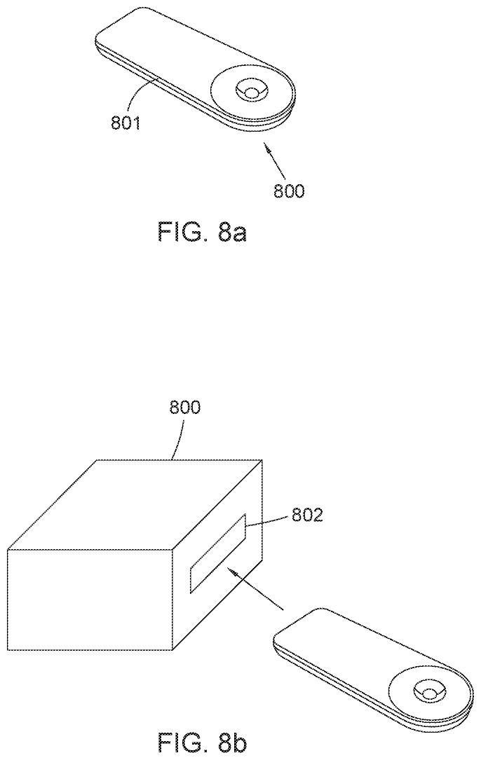

FIG. 8a illustrates an assay reader device comprising the optical module and a lateral flow test strip; and

FIG. 8b illustrates an assay reader device comprising the optical module and an aperture for receiving a lateral flow assay device comprising a lateral flow test strip.

### DETAILED DESCRIPTION OF THE PREFERRED EMBODIMENTS

Embodiments will now be described by way of example only with reference to the accompanying figures.

Lateral flow assays or other types of assays indicate the presence of a target molecule by the change of colour characteristics of a test region of the assay. FIG. 1 is a plot illustrating a known technique whereby light emitted by a visible light source is used to illuminate a test region of the assay. The light emitted by the visible light source has an excitation spectrum 102 centred on an excitation wavelength λ1 in the visible wavelength range. We refer herein to the visible light as being in the 380-700 nm range and near-infrared (NIR) being in the 700-2500 nm range. Known techniques use dyes (conjugates) in the visible wavelength range for detection since it is visible to the naked eye. FIG. 1 illustrates an autofluorescence spectrum 104 of one or more natural compounds centred on a wavelength λ2 in the visible wavelength range and an emission spectrum 106 of a desired dye centred on a wavelength λ3 in the visible wavelength range. As can be seen the autofluorescence spectrum 104 and the emission spectrum 106 of the desired dye overlap and thus the autofluorescence spectrum 104 acts as background noise in the emission measurement of the desired dye.

As noted above, the inventors have identified that other natural compounds exhibit autofluorescence at wavelengths within the visible range. For example, for natural compounds, such as human skin, blood, urine, plasma, nitrocellulose membrane, the autofluorescence emission is in the visible range 400 nm to 600 nm when excited with a shorter wavelength 300 nm to 500 nm. This autofluorescence (coming from non-dye or non-marker related material) acts as background noise with respect to the marker signal. Embodiments of the present disclosure are directed to reducing this background noise.

FIG. 2a illustrates a schematic block diagram of an optical module 100. A substrate 11 holds an optical detector 12, at least one near infra-red (NIR) light source 13, and an electrical signal processor 5 which is electrically coupled to an electrical output of the optical detector 12. The electrical signal processor 5 and the optical detector 12 may be parts of the same processing unit for example parts of the same application-specific integrated circuit (ASIC). The substrate 11 is placed adjacent to a lateral flow test strip 15, which includes test zones 6 and 7. Each one of test zones 6 and 7 are capable of binding a predetermined number (for example three) tagged analytes.

FIG. 2b illustrates example optical filters which covers the optical detector 12. The filter illustrated in FIG. 2b includes four different zones: three filters which transmit three different parts of the optical spectrum and a fourth part which is transparent to a broad range of wavelengths including those of the three filters for providing a reference signal.

FIG. 2c illustrates the optical detector behind the optical filters of FIG. 2b, whereby at least four different zones corresponding to the four sections of the optical filters can be detected, but the resolution is typically higher than the four zones of the filters. An array of sensors can be used, or a single sensor which can spatially resolve the transmitted light. It is envisaged that the number of filter zones may correspond to or be larger than the number of tagged analytes. In this way, scalable multiplexing capabilities for any number of analytes may be provided without the need for additional detectors. The electrical signal processor 5 comprises processing logic for processing the detected signal. The processing logic can use a reference threshold to provide a binary outcome, or alternatively be able to quantify the strength of the signal.

FIG. 3 illustrates the schematic cross section of FIG. 2a in a perspective view, showing additional optional structural features. As in FIG. 2a, the substrate 11 holds an optical detector 12, and at least one light NIR light source 13 for illuminating analytes 14 present on the lateral flow test strip 15, which may be for example a nitrocellulose paper strip. The substrate 11 may be a printed circuit board (PCB) (that is, a standalone PCB that may be provided in addition to any PCB of an assay reader device into which the optical module is incorporated as will be described below, or it may be integral with and a portion of the PCB of the assay reader device itself).

The at least one NIR light source 13 emits NIR light in the 700-2500 nm wavelength range. The at least one NIR light source 13 may be a pulsed or continuous light source.

Arranged on the substrate 11 is also one or more walls 16 which divide the space between the substrate 11 and the lateral flow test strip 15 into a plurality of adjoining sections, and which may fully or partially enclose the one or more light sources 13 and optical detector 12 to shield the optical detector 12 from light outside of the walls 16. The one or more walls 16 may optionally comprise light absorbing material to reduce unwanted noise caused by e.g. stray reflections inside the walls 16.

One or more of the walls 16 may comprise an aperture 17 to provide an optical path from the at least one light source 13 and optical detector 12 inside the walls 16 to the lateral flow test strip 15 outside the walls 16. The number of apertures 17 may determine how many test lines or zones may be simultaneously read. Where multiple apertures 17 are present, it is envisaged that multiple light sources 13 may be used. In the non-limiting example of FIG. 3, there are two apertures 17 and corresponding light sources 13 to read simultaneously two lines on the lateral flow test strip 15. Other numbers of apertures and corresponding light sources 13 are also envisaged, such as three, four, five, and more. In this way, even if a lateral flow test strip 15 has multiple test lines or zones with different illumination requirements, they may still be read simultaneously, namely through the use of multiple apertures 17, light sources 13, and/or the optical filters described above in relation to FIG. 2a.

Alternatively and/or additionally, one or more of the walls 16 may be arranged to block a portion of the field of view of the detector 12. For example, a wall 16a may be positioned between the optical detector 12 and the light source 13 so that the light source is not in the direct field of view of the optical detector 12. Instead light from the light source 13 only indirectly reaches the optical detector 12 through reflections and/or emissions from the lateral flow test strip 15. This ensures the optical detector 12 is not swamped by direct illumination and noise is thereby reduced.

Alternatively and/or additionally, in the case where multiple apertures 17 are present, one or more of the walls 16b may be arranged to prevent light from one aperture 17 interfering with light from the others at the optical detector 12, which may otherwise cause unwanted noise. For example, the walls 16 may be arranged such that the optical path from one aperture 17 does not intersect that of another. The walls 16 are thus arranged to control what light from different apertures 17 reaches different spatially separated regions of the optical detector 12.

One or more optical filter 10 is used in the detection of the presence of an analyte 14 on the test lines or zones on the lateral flow test strip 15. For multi-analyte detection, multiple optical filters 10 are used to discriminate between a plurality of different possible changes of the test line of the assay. The optical filter(s) 10 may be external to the optical detector 12, or the optical detector 12 may be wavelength sensitive and thereby include the optical filter(s) 10. The optical detector can be an array of photodiodes, whereby one or more of the photodiodes may have a corresponding optical filter provided in front thereof to thereby control what wavelength of light is received by the respective photodiode. One or more photodiodes may also be provided with a clear filter C or no filter. The array of photodiodes can be part of one or more ASICS.

The optical detector 12 is arranged with respect to the test region such that the test region is in the field of view of the optical detector 12. The NIR light source 13 may be arranged outside the field of view of the optical detector 12 to minimise noise that might otherwise be caused by direct illumination of the optical detector 12 with the light source 13. Additionally, or alternatively, noise caused by the reflectance of areas around the test and control lines on the lateral flow test strip can be reduced by minimising this reflectance. This may be achieved, for example, by arranging one or more optical components such as diaphragms, slits, walls, and/or other blocks in the optical path between the test region and the optical detector to reduce and/or block undesired light reflected from the areas around the test and control lines from reaching the optical detector. The test region may be on-axis or off-axis for the field of view of the detector. A planar optical detector may be used. For the optical detector 12 is it possible to use silicon, Si, (700-1150 nm); indium gallium arsenide, InGaAs, (−1600 nm); or germanium, Ge, and germanium-tin (1.4 um-2.4 um).

The test region of the assay may be a flow membrane with reaction regions, for example reaction lines, but the reaction region on the membrane may also be in the form of a circle, dot, or any other shape. Moreover, the reaction region can be a matrix of dots or can be referred to in general as test sites. The test region, which can accommodate multiple analytes, combined with the array of different optical filters enables simultaneous detection of multiple analytes. The signal can also be time resolved to detect reaction dynamics.

FIG. 4 is a flow diagram illustrating a method 400 for reading a test region of an assay in accordance with embodiments of the present disclosure. The method 400 comprises a step S402 of illuminating the test region with the NIR light source 13 that is operable to emit light in the NIR spectrum. At step S404 the test region of the assay is provided in the field of view of the optical detector 12. At step S406 light emitted from the test region is filtered using one or more optical filter 10 to provide filtered light. At step S408, the filtered light is detected with the optical detector 12.

Exemplary configurations of the above techniques will now be described. These configurations are not intended to be limiting and it is envisaged that elements of each configuration may be combined with each other.

In order to reduce background noise, one or more of three methods may be employed by the optical module 100 to avoid background noise caused by autofluoroescence:
1. Measuring absorption of a NIR conjugate (dye) using a NIR light source and NIR optical detector
2. Measuring NIR fluorescence emission of a NIR conjugate (dye) using a NIR light source and NIR optical detector.
3. Measuring light emission of reverse stoke shift conjugate (dye) using a NIR light source and either a visible light optical detector or a NIR optical detector.

FIG. 5a shows a plot associated with a test line of a lateral flow test strip 15 that comprises a conjugate (e.g. a fluorescent dye) that absorb light emitted by the NIR light source 13 as an excitation.

In particular FIG. 5a shows an excitation spectrum 502 of light emitted by the NIR light source 13. The excitation spectrum 502 is in the NIR wavelength range.

FIG. 5a also shows an example absorption spectrum 504 centred on a wavelength λ4 in the NIR wavelength range which is exhibited by the particular conjugate of the test line, the intensity of which is dependent on the amount of analyte present in the conjugate (the analyte will influence the concentration level). FIG. 5a shows three absorption spectrums with different intensities which may be detected from different sample fluids. Higher absorption of the NIR light results in lower reflection of NIR light from the test line. Similarly, lower absorption of the NIR light results in higher reflection of NIR light from the test line. An analyte can therefore be detected by a reduction rather than an increase in reflection.

When the sample region is illuminated with the excitation spectrum 502 of light emitted by the NIR light source 13 the sample will emit light at one or more longer wavelengths than the excitation wavelength (when a downconverting dye is used). FIG. 5a also shows an example emission spectrum 506 centred on a wavelength λ5 in the NIR wavelength range which is exhibited by the conjugate of the test line, the intensity of which is dependent on the amount of analyte present in the conjugate. As shown, the emission spectrum 506 is energetically lower than the excitation spectrum 502. FIG. 5a shows three emission spectrums with different intensities which may be detected from different sample fluids.

FIG. 5b shows a plot associated with a control line of the lateral flow test strip 15 that comprises a conjugate (e.g. a fluorescent dye) that absorb light emitted by the NIR light source 13 as an excitation. FIG. 5b shows an absorption spectrum 508 and emission spectrum 510 exhibited by the conjugate of the control line in response to NIR light being incident on the conjugate, the intensity of both will remain substantially constant during the detection of different sample fluids. Typically, the same conjugate dye is used for both the test and control line. Thus in these cases, the curves 504 and 508 will be identical. The test line will vary in concentration (due to varying amounts of analyte present), whereas the control line will not vary in concentration (it is either a constant −1 signal or 0 signal).

The first method described above uses absorption/reflection of light. The test region is illuminated with the NIR light source 13 and the reflected spectrum and its intensity (quantification) depends on the presence of analytes.

FIG. 5c illustrates an example filter response 512 associated with one or more optical filter 10 when employing the first method.

As shown in FIG. 5a, in embodiments directed to the first method the excitation spectrum 502 of light emitted by the NIR light source 13 is within (partially or completely) the absorption spectrum 504 exhibited by the conjugate of the test line. Expressed another way, the NIR light source 13 emits light at wavelengths that will be absorbed by the conjugate used in the assay.

As shown in FIG. 5c the filter response 512 has a pass-band that includes wavelengths of light emitted by the NIR light source 13 which will be absorbed by the conjugate. That is, the optical filter(s) are transparent (will not block) wavelengths of light emitted by the NIR light source 13 which will be absorbed by the conjugate of the assay. This enables the optical detector 12 to detect and quantify the amount of analyte present based on measuring the amount of emitted light that is reflected back from the conjugate to the optical detector 12 (where higher absorption of the NIR light results in lower reflection of the NIR light).

As illustrated in FIG. 5c, is it desirable to select NIR light source 13 that emits light in an excitation spectrum 502 at the flank of the absorption spectrum 504 to reduce the noise caused by the emission spectrum 506 exhibited by the conjugate being detected by the optical detector 12. When the NIR light source 13 emits light in an excitation spectrum 502 at the flank of the absorption spectrum 504, the excitation wavelength advantageously has no overlap with the emission wavelength.

The filter response 512 may have a stop-band that includes wavelengths of light in the emission spectrum 506 exhibited by the conjugate. That is, the optical filter(s) are configured to block wavelengths of light in the emission spectrum 506 exhibited by the conjugate. In these implementations, the one or more optical filter 10 may be configured as low-pass or band pass filters.

When multiple different analytes are present, multiple different absorption spectrums can be monitored. This is illustrated in FIG. 6. The plot 600 shown in FIG. 6 shows a broad excitation spectrum 602 of light emitted by the NIR light source 13, a first absorption spectrum 604 which is centred on a wavelength λ4 in the NIR wavelength range which is exhibited by the conjugate of the test line due to the presence of a first analyte, a second absorption spectrum 606 which is centred on a wavelength λ5 in the NIR wavelength range which is exhibited by the conjugate of the test line due to the presence of a second analyte, and a third absorption spectrum 608 which is centred on a wavelength λ6 in the NIR wavelength range which is exhibited by the conjugate of the test line due to the presence of a third analyte. In this example the optical filter(s) may comprise a first optical filter configured to be transparent to wavelengths associated with the first absorption spectrum 604 and block all other wavelengths, a second optical filter configured to be transparent to wavelengths associated with the second absorption spectrum 606 and block all other wavelengths, and a third optical filter configured to be transparent to wavelengths associated with the third absorption spectrum 608 and block all other wavelengths.

In the first method, the processing logic of the electrical signal processor 5 measures a signal indicative of the amount of absorption of light due to an analyte in the conjugate. The processing logic can use a reference threshold to provide a binary outcome, whereby a positive test result is provided if the measured signal is above the threshold (noting that low reflection corresponds to high absorption which is indicative that a target analyte is present) and whereby a negative test result is provided if the measured signal is below the threshold (noting that high reflection corresponds to low absorption which is indicative that a target analyte is not present). However, the processing logic is alternatively able to quantify the strength of the signal.

The second method described above uses fluorescence. As noted above when the sample region is illuminated with the excitation spectrum 502 of light emitted by the NIR light source 13 the sample will emit light at one or more longer wavelengths than the excitation wavelength (when a downconverting dye is used).

FIG. 5d illustrates an example filter response 514 associated with one or more optical filter 10 when employing the second method.

As shown in FIG. 5d the filter response 514 has a pass-band that includes wavelengths of the emission spectrum 506 exhibited by the conjugate. The filter response 512 may have a stop-band that includes wavelengths of light in the excitation spectrum 502 of light emitted by the NIR light source 13. That is, the optical filter(s) are configured to block wavelengths of light in the excitation spectrum 502 of light emitted by the NIR light source 13. When multiple different analytes are present, one or more excitation wavelengths can be used and multiple different emission wavelengths can be monitored.

In these implementations, the one or more optical filter 10 may be configured as high-pass or band pass filters.

In the second method, the processing logic of the electrical signal processor 5 takes a fluorescence measurement of the signal output by the optical detector 12. The processing logic can use a reference threshold to provide a binary outcome, whereby a positive test result is provided if the measured signal is above the threshold and whereby a negative test result is provided if the measured signal is below the threshold. However, the processing logic is alternatively able to quantify the strength of the signal.

A small disadvantage with the first method over the second method is a slight loss of dynamic range but with an increase of signal-to-noise ratio. The loss in dynamic range is due to the excitation wavelength of the excitation spectrum 502 of light emitted by the NIR light source 13 being at the flank of the absorption spectrum 504 compared to being at the peak of the absorption spectrum 504. The inventors have identified that by using near-infrared fluorescent dye that has a much more distinctive difference between absorption wavelength and emission wavelength, the loss of dynamic range can be mitigated.

When using the first method or the second method the NIR light source does not excite the autofluorescence of materials within the assay. Furthermore the optical detector 12 detects wavelengths of filtered light that are distant from the autofluorescence wavelengths of materials within the assay. This reduces the background noise and increases the analytical sensitivity of the measurements performed by the electrical signal processor 5.

Whilst in the second method referred to above a downconverting dye is used, in the third method an upconverting dye is used such when the sample region is illuminated with the excitation spectrum 502 of light emitted by the NIR light source 13 the sample will emit light at one or more shorter wavelengths than the excitation wavelength. In the third method the optical module 100 acts an anti-stoke fluorescent reader.

FIG. 7 shows a plot associated with a test line of a lateral flow test strip 15 that comprises a conjugate (e.g. a fluorescent dye) that absorb light emitted by the NIR light source 13 as an excitation. In particular FIG. 7 shows an excitation spectrum 702 of light emitted by the NIR light source 13. The excitation spectrum 702 is in the NIR wavelength range.

The excitation spectrum 702 is centred on an excitation wavelength λ1 in the NIR wavelength range which in the example shown in FIG. 7 is at the peak of an absorption spectrum 704 of the conjugate.

When the sample region is illuminated with the excitation spectrum 702 of light emitted by the NIR light source 13 the sample will emit light at one or more shorter wavelengths than the excitation wavelength (when an upconverting dye is used). FIG. 7 also shows an example emission spectrum 706 centred on a wavelength λ2 which is exhibited by the conjugate of the test line, the intensity of which is dependent on the amount of analyte present in the conjugate. As shown, the emission spectrum 706 is energetically lower than the excitation spectrum 502. FIG. 7 shows two emission spectrums with different intensities which may be detected from different sample fluids.

When employing the third method, the optical filter(s) are configured to be transparent to wavelengths associated with the emission spectrum 706 exhibited by the conjugate and block all other, or at least the excitation wavelengths.

In the third method, the processing logic of the electrical signal processor 5 takes a fluorescence measurement of the signal output by the optical detector 12. The processing logic can use a reference threshold to provide a binary outcome, whereby a positive test result is provided if the measured signal is above the threshold and whereby a negative test result is provided if the measured signal is below the threshold. However, the processing logic is alternatively able to quantify the strength of the signal.

When using the third method the NIR light source does not excite the autofluorescence of materials within the assay. Furthermore the emission spectrum 706 may be in the near-infrared spectrum such that the optical detector 12 detects wavelengths of filtered light that are distant from the autofluorescence wavelengths of materials within the assay. The emission spectrum 706 may also be in the visible spectrum, even in these implementations the fluorescence emission is too weak to trigger much autofluorescence, or it is negligible. Thus the third method also reduces the background noise and increases the analytical sensitivity of the measurements performed by the electrical signal processor 5.

In some embodiments of the present disclosure, the optical module 100 described herein is incorporated into an assay reader device 800 shown in FIG. 8a. As shown in FIG. 8a, the assay reader device 800 comprises an assay reader housing 801 housing the optical module 100 and the lateral flow test strip 15. Other components such as a printed circuit board, and power supply such as a battery may also be provided. The optical module may be secured to the printed circuit board of the assay reader device 800. The optical module may be the stand alone module with its own separate housing and substrate, or the optical module housing and/or substrate 11 may be integral with and/or form part of the assay reader housing 801 and printed circuit board. Mounted on the printed circuit board may also components to enable communications with an external device. For example, Bluetooth, Wi-Fi, USB and/or other wired and/or wireless communications components may be mounted on the printed circuit board in order to provide a network interface to communicate a result of the assay test to an external device, such as a mobile device, computer, cloud servers and the like.

In other embodiments of the present disclosure, the optical module 100 described herein is incorporated into an assay reader device 800 shown in FIG. 8b. In these embodiments, the assay reader device 800 does not comprise the lateral flow test strip 15. Instead, the assay reader device 800 comprises an aperture 802 (i.e. a slot/opening) for receiving a lateral flow assay device comprising a lateral flow test strip 15.

Although the disclosure has been described in terms of preferred embodiments as set forth above, it should be understood that these embodiments are illustrative only and that the claims are not limited to those embodiments. Those skilled in the art will be able to make modifications and alternatives in view of the disclosure which are contemplated as falling within the scope of the appended claims. Each feature disclosed or illustrated in the present specification may be incorporated in any embodiments, whether alone or in any appropriate combination with any other feature disclosed or illustrated herein.

### LIST OF REFERENCE NUMERALS

100 optical module
102 excitation spectrum centred on an excitation wavelength λ1 in the visible wavelength range
104 autofluorescence spectrum of one or more natural compounds centred on a wavelength λ2 in the visible wavelength range
106 emission spectrum 106 of a dye centred on a wavelength λ3 in the visible wavelength range
5 electrical signal processor
6 test zone
7 test zone
10 optical filter(s)
11 substrate
12 optical detector
13 NIR light source
14 analytes
15 lateral flow test strip
16 walls
17 aperture
502 excitation spectrum of light emitted by NIR light source
504 absorption spectrum of a test line of a lateral flow test strip
506 emission spectrum of a test line of a lateral flow test strip
508 absorption spectrum of a control line of a lateral flow test strip
510 emission spectrum of a control line of a lateral flow test strip
512 filter response
514 filter response
602 excitation spectrum of light emitted by NIR light source
604 first absorption spectrum
606 second absorption spectrum
608 third absorption spectrum
702 excitation spectrum of light emitted by NIR light source
704 absorption spectrum
706 emission spectrum
800 assay reader device
801 assay reader housing
802 aperture

1. An optical module for reading a test region of an assay, the optical module comprising:
a near-infrared light source for illuminating the test region of the assay with light in a near-infrared spectrum;
an optical detector, comprising an optical input for receiving light emitted from the test region of the assay and an electrical output;
an electrical signal processor, electrically coupled to the electrical output; and
one or more optical filter arranged in front of the optical input of the optical detector.

2. The optical module of claim 1, wherein the optical module comprises a plurality of optical filters.

3. The optical module of claim 2, wherein the plurality of optical filters correspond to a plurality of spatially separated regions of the optical detector.

4. The optical module of claim 2, wherein the optical detector comprises an array of detectors, and wherein each detector of the array of detectors corresponds to each of said optical filters.

5. The optical module of claim 1, wherein the one or more optical filter are arranged in front of the optical input of the optical detector such that there is no optical filter in front of a portion of the optical input.

6. The optical module of claim 1, wherein the optical detector comprises said one or more optical filter.

7. The optical module of claim 1, comprising a second near-infrared light source for illuminating a control region of the assay.

8. The optical module of claim 1, wherein the light emitted by the near-infrared light source has an excitation spectrum centred on an excitation wavelength and the one or more optical filter is transparent to at least a portion of said excitation spectrum, wherein said portion of the excitation spectrum is within an absorption spectrum of a conjugate used in said assay.

9. The optical module of claim 8, wherein the one or more optical filter block light having wavelengths within an emission spectrum of said conjugate.

10. The optical module of claim 1, wherein the light emitted by the near-infrared light source has an excitation spectrum centred on an excitation wavelength and the one or more optical filter is transparent to wavelengths within an emission spectrum of a conjugate used in said assay.

11. The optical module of claim 10, wherein said wavelengths within the emission spectrum of the conjugate used in said assay are at a higher wavelength than the excitation spectrum.

12. The optical module of claim 10, wherein said wavelengths within the emission spectrum of the conjugate used in said assay are at a lower wavelength than the excitation spectrum.

13. The optical module of claim 12, wherein said wavelengths within the emission spectrum of the conjugate used in said assay are in the visible spectrum.

14. The optical module of claim 12, wherein said wavelengths within the emission spectrum of the conjugate used in said assay are in the near-infrared spectrum.

15. The optical module of claim 10, wherein the one or more optical filter block light having wavelengths within the excitation spectrum of the infrared light source.

16. The optical module of claim 10, further comprising a substrate for mounting the near-infrared light source and the optical detector.

17. An assay reader device comprising the optical module of claim 1.

18. The assay reader device of claim 17 wherein the assay reader device comprises a lateral flow test strip.

19. The assay reader device of claim 17 wherein the assay reader device comprises an aperture for receiving a lateral flow assay device comprising a lateral flow test strip.

20. A method for reading a test region of an assay, the method comprising:
illuminating the test region with a near-infrared light source that is operable to emit light in a near-infrared spectrum;
providing the test region of the assay in the field of view of an optical detector;
filtering light emitted from the test region using one or more optical filter to provide filtered light; and
detecting the filtered light with the optical detector.
